(19) United States
(12) Patent Application Publication
(10) Pub. No.: US 2002/0160413 A1
(43) Pub. Date: Oct. 31, 2002

(54) COMBINATORIAL LIBRARIES

(76) Inventors: BENJAMIN L. MILLER, ROCHESTER, NY (US); BRYAN KLEKOTA, VICTOR, NY (US)

Correspondence Address: PETER ROGALSKYJ, NIXON HARGRAVE DEVANS & DOYLE, CLINTON SQUARE, P O BOX 1051, ROCHESTER, NY, 14603

(21) Appl. No.: 09/181,108

(22) Filed: Oct. 28, 1998

(51) Int. Cl. G01N 33/53; C07F 3/06

(52) U.S. Cl. 435/7.1; 548/402

(57) ABSTRACT

The present invention relates to a combinatorial library which includes a plurality of at least six different complexes. Each of the complexes is formed of at least one complexing agent and at least two non-biopolymer ligands that are reversibly bonded to the complexing agent, and each different complex in the library has different ligands bonded to the complexing agent. Compositions that include these combinatorial libraries and receptors are also disclosed, as are methods for identifying a combination of non-biopolymer ligands which bind preferentially to a receptor. Methods for producing the combinatorial libraries are also described. The combinatorial libraries, compositions, and methods of the present invention permit the selection and amplification of non-biopolymeric molecules which are targeted to a particular receptor, where the selection and amplification criteria are based strictly on differences in binding affinity to a receptor. Thus, the present invention has utility for the identification and preparation of non-biopolymeric molecules which are targeted to a particular receptor.

This work was supported by the National Science Foundation, Grant No. CHE-9322203. The Federal Government may have certain rights in the invention.

### FIELD OF THE INVENTION

The present invention relates to combinatorial libraries and to methods for making and using same.

### BACKGROUND OF THE INVENTION

Until recently, the development of a ligand for a biological receptor has required the iterative synthesis, testing, and modification of individual compounds based on natural product leads or de novo molecular design. The emerging field of combinatorial synthesis shows great promise for streamlining this process, enabling the rapid generation of structurally diverse libraries of up to millions of compounds, which may be screened against a biological receptor (Gordon et al., “Applications of Combinatorial Technologies to Drug Discovery. 2. Combinatorial Organic Synthesis, Library Screening Strategies, and Future Directions” J. Med. Chem., 37:1385-1401 (1994); Virgilio et al., “Simultaneous Solid-Phase Synthesis of beta-Turn Mimetics Incorporating Side-Chain Functionality” J. Am. Chem. Soc., 116:11580-11581 (1994); Brenner et al., “Encoded Combinatorial Chemistry” Proc. Natl. Acad. Sci. USA, 89:5381-5383 (1992); Ohlmeyer et al., “Complex Synthetic Chemical Libraries Indexed With Molecular Tags” Proc. Natl. Acad. Sci. USA, 90:10922-10926 (1993); Carell et al., “New Promise in Combinatorial Chemistry: Synthesis, Characterization, and Screening of Small-Molecule Libraries in Solution,” Chemistry & Biology, 2:171-183 (1995)).

However, this approach does not represent a complete solution to the problem of identifying receptor ligands, for several reasons.

First, unless the library is designed to “tune” the activity of a lead compound, the proportion of active species relative to the whole is extremely small. This has the effect of rendering detection of the active compounds against the background of inactive compounds problematic, since standard methods of measuring a binding constant are not meaningful when applied to mixtures of compounds. Typically, the detection problem has been addressed by coupling the receptor to a reporter molecule, such as a dye, a fluorescent tag, or a substrate for another, readily detectable protein (e.g., coupling to biotin for detection by a streptavidin-alkaline phosphatase conjugate). However, these techniques, particularly the two-stage assay process, can change the properties of the receptor of interest, or cause ligands to the secondary receptor rather than the primary receptor to be identified (Devlin et al., “Random Peptide Libraries: A Source of Specific Protein Binding Molecules” Science 249:404-406 (1990); Lam et al., “A New Type of Synthetic Peptide Library for Identifying Ligand-Binding Activity” Nature (London), 354:82-84 (1991)).

Second, the total amount of active compound present in an assay of this type is generally too small to permit direct structural analysis by spectroscopic means, requiring instead that the compound be identified through the analysis of encoded tags or iterative deconvolution (although some direct methods employing gel-phase NMR or mass spectrometry (Youngquist et al., “Generation and Screening of Combinatorial Peptide Libraries Designed for Rapid Sequencing by Mass Spectrometry” J. Am. Chem. Soc 117:3900-3906 (1995) have appeared), and subsequent resynthesis for full characterization. Finally, if library synthesis and evaluation is carried out on solid support, compound behavior is not necessarily transferable to the solution phase. On the other hand, solution-based strategies for library synthesis require that analysis proceed through the time-consuming process of iterative deconvolution (Erb et al., “Recursive Deconvolution of Combinatorial Chemical Libraries” Proc. Natl. Acad. Sci. USA, 91:11422-11426 (1994)).

The present invention is directed to overcoming these and other deficiencies in the art.

### SUMMARY OF THE INVENTION

The present invention relates to a combinatorial library which includes a plurality of at least six different complexes. Each of the complexes is formed of at least one complexing agent and at least two non-biopolymer ligands that are reversibly bonded to the complexing agent, and each different complex in the library has different ligands bonded to the complexing agent.

The present invention also relates to a composition that includes a combinatorial library and a receptor in contact with the combinatorial library. In this composition, some of the complexes bind preferentially to the receptor. The combinatorial library includes a plurality of at least six different complexes. Each of the complexes is formed of at least one complexing agent and at least two non-biopolymer ligands that are reversibly bonded to the complexing agent, and each different complex in the library has different ligands bonded to the complexing agent.

The present invention also relates to a method for identifying a combination of non-biopolymer ligands which bind preferentially to a receptor. The method includes providing a combinatorial library; contacting the combinatorial library with a receptor under conditions effective to preferentially bind a fraction of the plurality of complexes; and identifying the fraction of the plurality of complexes which are bound preferentially to the receptor. In this method, the combinatorial library includes a plurality of at least six different complexes. Each of the complexes is formed of at least one complexing agent and at least two non-biopolymer ligands that are reversibly bonded to the complexing agent, and each different complex in the library has different ligands bonded to the complexing agent.

The present invention also relates to a methods for producing a combinatorial library which includes a plurality of at least six different complexes. Each of the complexes in the library produced by these methods is formed of at least one complexing agent and at least two non-biopolymer ligands that are reversibly bonded to the complexing agent, and each different complex in the library has different ligands bonded to the complexing agent. Furthermore, each of the plurality of complexes has the formula Z(Aⁱ)ₙ, where Z is a complexing agent capable of reversibly binding to two or more ligands, each Aⁱ is a non-biopolymer ligand capable of reversibly binding to Z and is independently selected from a group of non-biopolymer ligands having at least three different members, n is the number of A's that are reversibly bonded to Z and is an integer equal to two or greater, and i is an index number for each A and is an integer from 1 to n.

In one method for producing a combinatorial library to which the present invention relates, one part of a complexing agent, Z, or a salt thereof is contacted with m¹ parts of A¹ and mⁱ parts of Aⁱ under conditions effective to form a substantially statistical mixture of Z(Aⁱ)ₙ. A¹ and each Aⁱ are non-biopolymer ligands; i is an integer from 2 to k; k is an integer equal to or greater than 3 and represents the number of members in the group of at least three different non-biopolymer ligands from which the at least two non-biopolymer ligands are selected; and the sum of m¹ and Σmⁱ from i=2 to k equals n.

In another method for producing a combinatorial library to which the present invention also relates, a complex, Z(A¹)ₙ is contacted with two or more complexes Z(Aⁱ)ₙ under conditions effective to form a substantially statistical mixture of Z(Aⁱ)ₙ. A¹ and each Aⁱ are non-biopolymer ligands; i is an integer from 2 to k; and k is an integer equal to or greater than 3 and represents the number of members in the group of at least three different non-biopolymer ligands from which the at least two non-biopolymer ligands are selected.

The combinatorial libraries, compositions, and methods of the present invention permit the selection and amplification of non-biopolymeric molecules which are targeted to a particular receptor, where the selection and amplification criteria are based primarily on differences in binding affinity to a receptor. Thus, the present invention has utility for the identification and preparation of non-biopolymeric molecules which are targeted to a particular receptor.

### BRIEF DESCRIPTION OF THE DRAWINGS

FIG. 1 is a bar graph showing the relative amounts of various ligands in initial elutions in the presence of various concentrations of zinc ion and in the presence and absence of salicylaldehyde.

FIG. 2 is a bar graph showing the relative amounts of various ligands in initial elutions from unfunctionalized cellulose resin in the presence of various concentrations of zinc ion and in the presence and absence of salicylaldehyde.

FIG. 3 is a graph showing the dependence of UV absorbance of a mixture of salicylaldehyde and N-methyl 2-aminoethyl pyrrolidine on the zinc ion concentration.

FIG. 4 is a bar graph showing the peak volumes for libraries prepared in the presence and absence of zinc ion.

FIG. 5 is a bar graph showing the effect of zinc ion presence on the peak area of various complexes contained in libraries according to the present invention eluted from DNA-cellulose.

FIG. 6 is a bar graph showing the effect of zinc ion presence on the peak area of various complexes contained in libraries according to the present invention eluted from control cellulose.

FIG. 7 is a schematic diagram showing a process according to the present invention.

FIG. 8 is a bar graph showing the results of affinity selection and amplification of a self-assembled combinatorial library of the present invention using a double-stranded DNA receptor.

FIG. 9 is a bar graph showing the results of affinity selection and amplification of a self-assembled combinatorial library of the present invention using a single-stranded DNA receptor on cellulose and the results of a control selection on cellulose alone.

FIG. 10 is a bar graph showing the ratio of a pair of complexes in a combinatorial library of the present invention following incubation with double-stranded DNA in the presence and absence of zinc.

FIG. 11 is a series of NMR spectra showing the effect of the ratio of a ligand to ZnCl₂ on the NMR spectrum.

### DETAILED DESCRIPTION OF THE INVENTION

The present invention relates to a combinatorial library which includes a plurality of at least six different complexes. Each of the complexes is formed of at least one complexing agent and at least two non-biopolymer ligands that are reversibly bonded to the complexing agent, and each different complex in the library has different ligands bonded to the complexing agent. Two ligands which are structurally the same but which have different optical configurations are to be considered “different” for purposes of the present invention.

As used herein, a complexing agent is meant to include any atom, group of atoms, or ion that is capable of reversibly binding two or more non-biopolymer ligands. Examples of suitable complexing agents include metal atoms and metal ions, which, as used herein, are meant to include Na, K, Rh, Cs, Fr, Mg, Ca, Sr, Ba, Ra, Sc, Y, the lanthanides, the actinides, Ti, Zr, Hf, V, Nb, Ta, Cr, Mo, W, Mn, Tc, Re, Fe, Ru, Os, Co, Rh, Ir, Ni, Pd, Pt, Cu, Ag, Au, Zn, Cd, Hg, B, Al, Ga, In, Tl, Si, Ge, Sb, Pb, P, As, Sb, Bi, Se, Te, Po, and their stable ions. Preferably, the metal or metal ion complexing agent has a valency such that it undergoes fast ligand exchange. Considerations for selecting such preferred metal or metal ion complexing agents based on this criterion are known to those of ordinary skill in the art, and further detail regarding these considerations is are set forth in Cotton et al., Advanced Inorganic Chemistry. A Comprehensive Text, 4th ed., New York: John Wiley & Sons (1980) (“Cotton”), which is hereby incorporated by reference. For example, since Co(II) undergoes fast ligand exchange while Co(III) does not, the use of Co(II) as a complexing agent is preferred to Co(III) in the practice of the present invention.

As used herein, a ligand is meant to include any compound that is capable of reversibly binding to the complexing agent. A non-biopolymer ligand is meant to include any ligand provided that the ligand is not a DNA molecule, an RNA molecule, or a polypeptide. For purposes of the present invention, DNA and RNA molecules are compounds that consist solely of two or more nucleotides linked by phosphodiester bonds, and polypeptides are compounds that consist solely of two or more amino acids linked by amide bonds. Other biologically based compounds, such as cellulose, starches, sugars, and the like are considered to be non-biopolymer compounds for the purposes of the present invention.

Non-biopolymer ligands suitable for use in the practice of the present invention are those which reversibly bind to the complexing agent. Where the complexing agent is a metal atom or metal ion, suitable ligands are those compounds that have at least one functional group capable of bonding to a metal or metal ion. Examples of such functional groups include hydroxyl groups, nitrogen-containing groups (including, e.g., amino groups and heterocyclic nitrogens), carboxyl groups (e.g., ketones, aldehydes, and carboxylic acids and derivatives thereof) and the like. Illustratively, the following structures represent suitable non-biopolymer ligands:

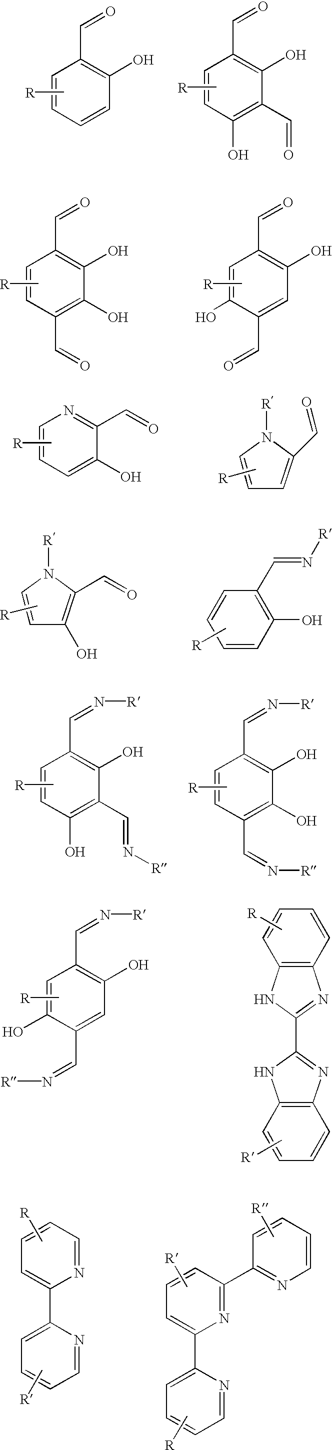

where R, R′, R″, and R′″, when bonded to a ring, represent one or more ring substituents and, when bonded to a nitrogen atom, represent a single substituent. Suitable ring substituents include, halogen, hydroxy, substituted or unsubstituted alkyl, alkoxy, aryloxy, substituted or unsubstituted aryl, an amino group (which may, optionally, be substituted with one or two alkyl or aryl groups), a carboxylic acid, ester, or amide group, and a substituted or unsubstituted heterocycle (e.g., a substituted or unsubstituted pyridyl, furanyl, piperidinyl, or morphilino group). Suitable nitrogen substituents include substituted or unsubstituted alkyl, substituted or unsubstituted aryl, or substituted or unsubstituted heterocycle (e.g., a substituted or unsubstituted pyridyl, furanyl, piperidinyl, or morphilino group).

Preferably the non-biopolymer ligand also contains one or more groups which are capable of recognizing a biological element, such as a DNA molecule or a particular portion of a DNA molecule. Such a group will be referred to herein as a recognition element. Examples of such recognition elements include DNA intercalators, such as those which have the following formulae:

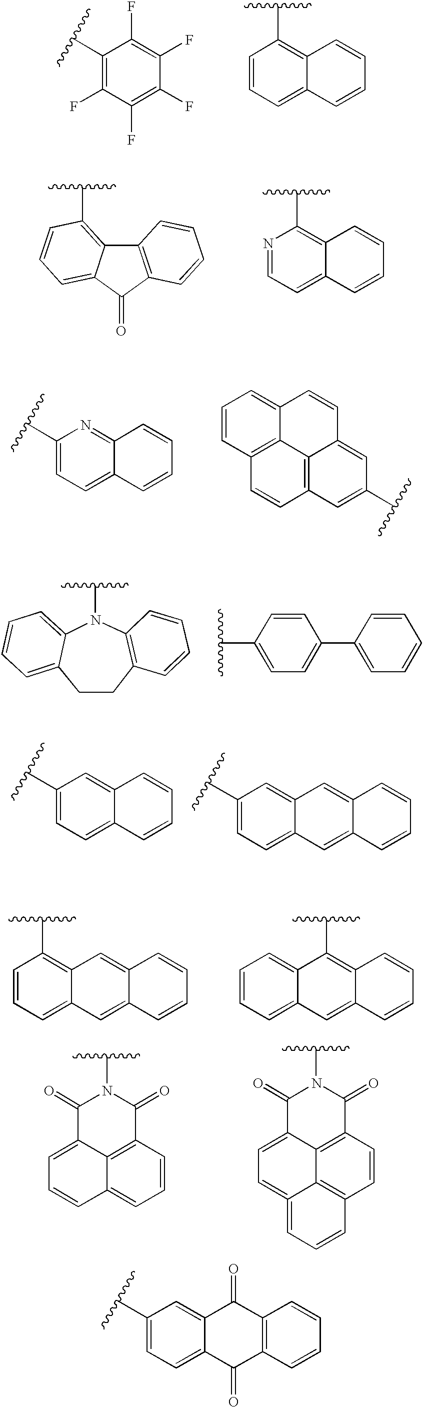

Preferred intercalators are pyrenes and anthracenes. Other examples of such recognition elements include DNA major or minor groove binders, such as those which have the following formulae:

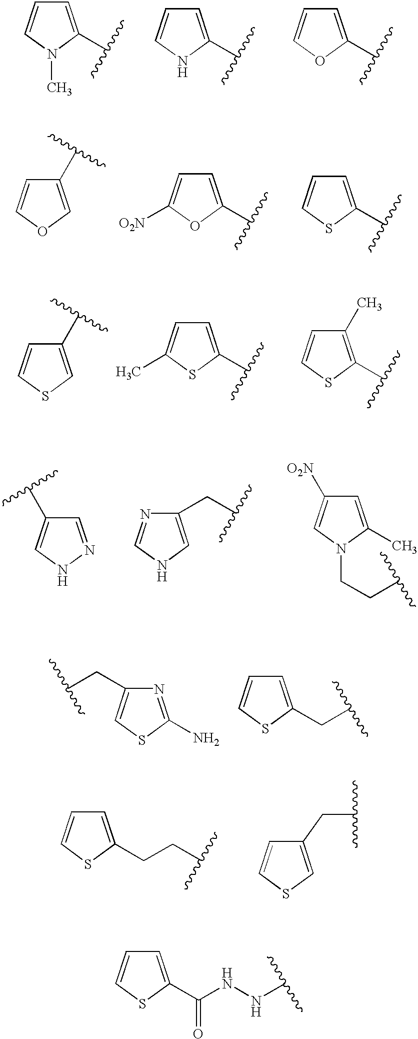

Other suitable recognition elements include hydroxy groups, pyrrolid-2-yl groups, N-alkylpyrrolid-2-yl (e.g., N-methylpyrrolid-2-yl) groups, alkoxy (e.g., methoxy) groups, tetrahydrofuran-2-yl groups, pyrid-2-yl groups, and substituted or unsubstituted phenyl (e.g., 4-fluorophenyl) groups.

These recognition elements can be bonded directly to the above-described non-biopolymer ligands as one or more of the recited R′, R″, and R′″ groups. Alternatively, they can be bonded through a suitable connector moiety to the above-described non-biopolymer ligands as one or more of the recited R′, R″, and R′″ groups. Suitable connectors include substituted or unsubstituted alkylene moieties (e.g., —CH₂—, —CH₂CH₂—, —CH₂CH₂CH₂—, —CH₂CH(CH₃)CH₂—). Other suitable connectors include moieties having the formulae:

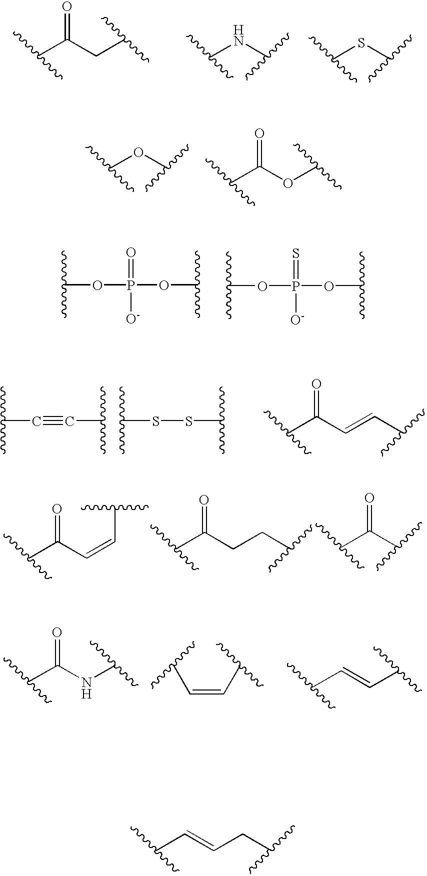

which may be optionally substituted (e.g., with alkyl or aryl groups) at the carbons or nitrogens to which hydrogens are bonded.

The above-discussed non-biopolymer ligands can be expressed in terms of the following formula:

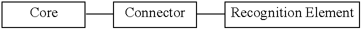

where the recognition element and connector are as described above and where suitable core moieties are those above-described structures which have one or more R, R′, R″, and R′″ groups bonded thereto. As one skilled in the art will appreciate, a core can be connected to more than one connector, and each connector may, in turn, be attached to one or more recognition elements.

Suitable non-biopolymer ligands also can include simple molecules, such as, for example, water, amines, alcohols, thiols, and carboxylic acids and derivatives thereof. Amines, alcohols, and thiols that can be used as non-biopolymer ligands in the practice of the present invention can be primary or secondary amines, alcohols, and thiols. Compounds containing two or more of these functional groups (e.g., ethylenediamine) can also be employed. Preferably, at least one of the non-biopolymer ligands contains a group that is capable of recognizing a biological element, such as a DNA molecule or a particular portion of a DNA molecule.

As indicated above, the combinatorial library of the present invention includes a plurality of at least six different complexes and each of the complexes is formed of at least one complexing agent and at least two non-biopolymer ligands that are reversibly bonded to the complexing agent. In addition, the combinatorial library includes at least six different complexes and each different complex in the library has different ligands bonded to the complexing agent.

For example, the combinatorial library can include a plurality of complexes, each of which has the formula Z(Aⁱ)ₙ, where Z is a complexing agent capable of reversibly binding to two or more ligands, each Aⁱ is a non-biopolymer ligand capable of reversibly binding to Z, and n is an integer equal to two or more and represents the number of non-biopolymer ligands that are bonded to Z. In the above formula, i is an index number which identifies each of the ligands individually (e.g., A¹, A², A³, A⁴, etc) and can have values from 1 to n. Each Aⁱ, is independently selected from a group of non-biopolymer ligands having at least three different members. Illustratively, when the number of non-biopolymer ligands that are bonded to Z is equal to 2, n will be 2, and there will be two A's (i.e., A¹ and A²); when the number of non-biopolymer ligands that are bonded to Z is equal to 3, n will be 3, and there will be three A's (i.e., A¹, A², and A³); when the number of non-biopolymer ligands that are bonded to Z is equal to 4, n will be 4, and there will be four A's (i.e., A¹, A², A³ and A⁴); when the number of non-biopolymer ligands that are bonded to Z is equal to 5, n will be 5, and there will be five A's (i.e., A¹, A², A³ , A⁴, and A⁵); when the number of non-biopolymer ligands that are bonded to Z is equal to 6, n will be 6, and there will be six A's (i.e., A¹, A², A³A⁴, A⁵, and A⁶); when the number of non-biopolymer ligands that are bonded to Z is equal to 7, n will be 7, and there will be seven A's (i.e., A¹, A², A³ A⁴ A, A , A⁶ and A⁷); and when the number of non-biopolymer ligands that are bonded to Z is equal to 8, n will be 8, and there will be eight A's (i.e., A₁, A₂, A₃ A₄, A₅, A₆ A₇, and A₈). Stated differently, each of the plurality of complexes can be said to have the formula Z(A¹)(A²)(Aⁱ)ₙ₋₂, where A¹ and A² are non-biopolymer ligands capable of reversibly binding to Z and are independently selected from a group of non-biopolymer ligands having at least three different members. In this case, (Aⁱ)ₙ₋₂ represents the remaining (i.e., other than A¹ and A²) n-2 non-biopolymer ligands that are bonded to Z.

As one skilled in the art will appreciate, the values of n and i, as used herein, are not to be construed as an indication of the total number of A's in the group (e.g., ligands in the library) from which the A's are selected. Instead, n refers to the number of ligands bonded to the complexing agent, and i is an index number for each of those ligands.

As indicated above, each non-biopolymer ligand is independently selected from a group of non-biopolymer ligands having at least three different members. For purposes of illustration, the at least three different members from which each non-biopolymer ligand is independently selected will be designated B1, B2, and B3. Thus, when n non-biopolymer ligands are bonded to Z, the combinatorial library can include complexes having the formulae Z(B1)(B1)(Aⁱ)ₙ₋₂, Z(B1)(B2)(Aⁱ)ₙ₋₂, Z(B1)(B3)(Aⁱ)ₙ₋₂, Z(B2)(B2)(Aⁱ)ₙ₋₂, Z(B2)(B3)(Aⁱ)ₙ₋₂, and Z(B3)(B3)(Aⁱ)ₙ₋₂. For example, when n is 2, the combinatorial library includes complexes having the formulae Z(B1)(B1), Z(B1)(B2), Z(B1)(B3), Z(B2)(B2), Z(B2)(B3), and Z(B3)(B3). While the minimum number of members in the group from which non-biopolymers are selected is 3, this number can be higher (e.g., 4, 5, 6, 7, 8, 9, 10, or more). For example, when the number of members in the group from which non-biopolymers are selected is 4 (e.g., B1, B2, B3, and B4) and the number of non-biopolymer ligands bound to each Z is 2, the combinatorial library can include complexes having the formulae Z(B1)(B1), Z(B1)(B2), Z(B1)(B3), Z(B1)(B4), Z(B2)(B2), Z(B2)(B3), Z(B2)(B4), Z(B3)(B3) Z(B3)(B4), and Z(B4)(B4). Generally when the number of members in the group from which non-biopolymers are selected is m (e.g., B 1, B2, B3, . . . Bm), and the number of non-biopolymer ligands bound to each Z is 2, the combinatorial library will contain (m²+m)/2 different complexes. As indicated above, the minimum number of non-biopolymer ligands which are bound to each Z is 2. However, depending on the nature of Z and of the non-biopolymer ligands present, the number of non-biopolymer ligands which are bound to each Z can be greater than 2 (e.g., 3, 4, 5, 6, 7, 8, or more). For purposes of the present invention, a ligand bonded to Z is counted only once, even if it is bonded through two or more groups (such as where the ligand is a bidentate, tridentate, or tetradentate ligand).

Also included in the meaning of non-biopolymer ligands, A, are moieties having the formula —A′Z′(Q), where A′ is a pluridentate (i.e., bidentate, tridentate, etc.) non-biopolymer ligand, Z′ is a second complexing agent that is identical to or different than Z, and Q represents one or more non-biopolymer ligands. Thus, for example, when each Z is bonded to two non-biopolymer ligands (i.e., n=2) selected from a group of ligands containing at least one pluridentate (i.e., bidentate, tridentate, etc.) ligand, A′, and other ligands, the combinatorial library of the present invention can contain complexes having the formula:

Q—Z—A′—Z—Q

where each Q is independently selected from the group of ligands (i.e., Q can be A′ or one of the other ligands in the group).

In the case where each Z is bonded to three non-biopolymer ligands (i.e., n=3) selected from a group of ligands containing at least one pluridentate (i.e., bidentate, tridentate, etc.) ligand, A′, and other ligands, the combinatorial library of the present invention can contain complexes having the formula:

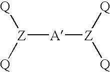

where each Q is independently selected from the group of ligands (i.e., Q can be A' or one of the other ligands in the group).

In still another embodiment of the present invention, two or more of the non-biopolymer ligands, A, can be moieties having the formula —A'Z'(Q), where each A' is the same or different and is a pluridentate (i.e., bidentate, tridentate, etc.) non-biopolymer ligand, each Z′ is a second complexing agent that is identical to or different than each other and Z, and Q represents one or more non-biopolymer ligands. Thus, for example, in the case where each Z is bonded to three non-biopolymer ligands (i.e., n=3) selected from a group of ligands containing at least one pluridentate (i.e., bidentate, tridentate, etc.) ligand, A′, and other ligands, the combinatorial library of the present invention can contain complexes having the formula:

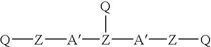

where each Q is independently selected from the group of ligands (i.e., Q can be A′ or one of the other ligands in the group) and each A′ can be the same or (in the case where the group of ligands contains more than one pluridentate ligand) different. Alternatively, when each Z is bonded to three non-biopolymer ligands (i.e., n=3) selected from a group of ligands containing at least one pluridentate (i.e., bidentate, tridentate, etc.) ligand, A′, and other ligands, the combinatorial library of the present invention can contain complexes having the formula:

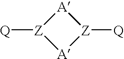

where each Q is independently selected from the group of ligands (i.e., Q can be A′ or one of the other ligands in the group) and each A′ can be the same or (in the case where the group of ligands contains more than one pluridentate ligand) different. Stated more generally, where the combinatorial library contains complexes having the formula Z(A¹)(A²)(A¹)ₙ₋₂, A¹ and A², taken together, can represent a moiety having the formula:

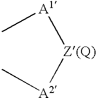

where A¹ and A^{2′} are pluridentate (i.e., bidentate, tridentate, etc.) ligands and are the same or (in the case where the group of ligands contains more than one pluridentate ligand) different. In these cases, Z(A¹)(A²)(Aⁱ)ₙ₋₂ has the formula:

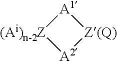

As indicated above, the non-biopolymer ligands, A, bound to the complexing agent, Z, are bound reversibly. Thus, for example, suitable complexes are those which can undergo ligand exchange. Preferred complexes are those where Z and each A are selected so that the reactions Z(A′)ₙ₋₁+A¹→Z(A¹)(A¹)ₙ₋₁ and Z(A¹)(A¹)ₙ₋₁→Z(A¹)ₙ₋₁+A¹ each have a rate constant of greater than about 2 per second, more preferably, greater than about 4 per second, and, most preferably, greater than about 10 per second. Rate constants for these reactions can be measured by conventional methods, which are well known to those skilled in the art, such as, for example, by the methods described in Cotton, particularly in Chapter 28, which is hereby incorporated by reference.

As indicated above the combinatorial libraries of the present invention contain at least six different complexes. For purposes of the present invention, two complexes are different if one complex contains a ligand which is not present in the other complex. As one skilled in the art will recognize the number of complexes in the combinatorial library will depend on the number of ligands present in the group of ligands from which the ligands are selected and on the number of ligands which can bind to a single complexing agent. Preferably, the combinatorial library contains at least about 20 different complexes; more preferably, the combinatorial library contains at least about 40 different complexes; still more preferably, the combinatorial library contains at least about 60 different complexes; still more preferably, the combinatorial library contains at least about 100 different complexes; still more preferably, the combinatorial library contains at least about 250 different complexes; and most preferably, the combinatorial library contains at least about 500 different complexes.

The present invention also relates to a composition which includes a combinatorial library and a receptor in contact with the combinatorial library. Combinatorial libraries suitable for the practice of the present invention include those described hereinabove. Suitable receptors include, for example, biological receptors, such as, DNA molecules, RNA molecules, oligosaccharides, whole cells or fragments thereof, proteins and other polypeptides, and polymers. Typically, the receptor will be one having particular biological interest, for example, a particular protein, RNA molecule, or DNA molecule that is linked to the occurrence of a particular disease, disease state, or condition. Many such proteins, RNA molecules, or DNA molecules are known to those of ordinary skill in the art.

The receptor is in contact with the combinatorial library. Typically, this is achieved by dissolving or suspending the constituent parts of the combinatorial library in a suitable medium (e.g., water or a buffered aqueous solution) which is in contact with the receptor. The receptor can be also dissolved or suspended in the medium or it can be immobilized on a solid support, such as cellulose or related resins. Advantageously, the receptor is immobilized on a solid support material that is packed into a column, and the combinatorial library is suspended or dissolved in a medium (e.g., water or a buffered aqueous solution) which is present in the column and in contact with the solid support material on which the receptor is immobilized. Immobilization of biological receptors on solid supports can be carried out using conventional methods, which are well known to those of ordinary skill in the art. As one of ordinary skill in the art will recognize, where temperature or light sensitive biological receptors are employed, appropriate measures should be taken to prevent denaturation of these biological materials. Upon initial contact of the receptor with the combinatorial library, few if any of the receptors will be bound by the complexes contained in the library; however, over time, an increasing number of the receptors will be bound by these complexes. Thus, the composition of the present invention may contain receptors which are bound to complexes contained in the combinatorial library. Generally, one skilled in the art will be able to predict the types of ligands which will bind to a particular biological receptor, and the combinatorial library will be designed accordingly. For example, where the biological receptor is a DNA molecule, one skilled in the art would identify DNA intercalators or major or minor groove binders as suitable ligands for construction of the combinatorial library.

The combinatorial libraries of the present invention are useful for identifying a combination of non-biopolymer ligands which binds preferentially to a receptor, such as a biological receptor, examples of which include those provided above. As used herein, the term “identifying” is meant to include “preparing” and “isolating” even though the identity of the desired combination may already be known.

The method includes providing a combinatorial library of the present invention, such as one of the combinatorial libraries described hereinabove, and contacting the combinatorial library with a receptor under conditions effective to preferentially bind a fraction of the plurality of complexes present in the combinatorial library to the receptor. Generally, the combinatorial library is present as a suspension or solution in water or some other suitable solvent, and the receptor is either dissolved or suspended in the water or other suitable solvent or immobilized, for example, on a suitable resin.

Contacting is carried out under conditions which are effective to preferentially bind a fraction of the plurality of complexes present in the combinatorial library. For example, the temperature at which the contacting is carried out is preferably between about 0° C. and 40° C., although other temperatures can be employed so long as the biological receptor is stable at these temperatures and so long the ligand exchange rates (i.e., the rate at which the reactions Z(Aⁱ)ₙ₋₁+A¹→Z(A¹)(Aⁱ)ₙ₋₁ and Z(A¹)(Aⁱ)ₙ₋₁→Z(Aⁱ)ₙ₋₁+A¹ occur) remain reasonably fast (preferably greater than about 2 per second). Physiologically relevant conditions (e.g., about 1-100 mM N-(2-hydroxytethyl)piperazine-N′(2-ethanesulfonic acid) (“HEPES”), about 10-1000 mM KCl, and pH from about 7 to about 8) are generally suitable conditions for carrying out the contacting step of the method of the present invention, especially where biological receptors are involved. The time period for which the combinatorial library and receptor is contacted can vary, but, generally, longer times give rise to better results in terms of the fraction of receptors that are bound to the preferentially-bound complex. Suitable time periods for maintaining contact between the combinatorial library and receptor are from about 10 minutes to about 5 days, preferably from about 1 hour to about 8 hours.

As a result of the above-described method, the receptors are preferentially bound (preferably, substantially exclusively bound) to the complex having the highest affinity (i.e., the greatest equilibrium constant) therefor. Most if not all other complexes remain unbound. A complex can be considered to be preferentially bound to a receptor if, after equilibrium is established, the complex occupies greater than about 50%, preferably, greater than about 75%, and more preferably, greater than about 90% of the available receptor sites.

Although not wishing to be bound by the following proposed explanation, applicants believe that the observed phenomenon can be explained by virtue of the fact that the binding between the ligands and the complexing agent is reversible, thus permitting ligands to exchange in response to perturbations in the ligand/complexing agent equilibrium. By adding a receptor, however, an additional equilibration ensues. This equilibration involves the binding of the complexes to the receptor with varying degrees of affinity. Since some complexes bind with higher affinity than others, those complexes would be depleted from the pool of equilibrating complexes. By simple mass balance rules, the equilibrium would then have to shift in favor of complexes that bind to the receptor, effectively using the thermodynamics of ligand-receptor binding to direct the synthesis of the complex having the highest affinity for the receptor. In the end, assuming there is sufficient amounts of the ligands necessary to produce a sufficient amount of highest affinity complex, the receptor is saturated with the highest affinity complex.

The preferentially bound complex can then be identified or characterized, such as by conventional methods (GC-MS, HPLC, etc). Typically, this involves separating the preferentially bound complex from the other complexes that are in the combinatorial library. Since the preferentially bound complex is attached to the receptor, this is generally achieved by separating the receptor (with preferentially bound receptor attached thereto) from the other complexes that are in the combinatorial library. In the cases where the receptor is immobilized, for example, as described above, such separation can be readily achieved, for example, by elution, filtration, and the like. In cases where the receptor is suspended or dissolved in a medium (e.g., a solvent) containing the other complexes in the combinatorial library, separation can be effected by filtration, polyethylene glycol precipitation, and the like. Once separation is achieved, the preferentially bound complex can be readily identified using conventional characterization methods. In some cases, it may be advantageous to separate the preferentially bound complex from the receptor to which it is bound. This can be effected by denaturation of the receptor or by elution from the receptor. Preferably, such a separation should be carried out under conditions which disfavor ligand exchange. In some situations, this can be achieved by changing the oxidation state of the metal complexing agent so that, after the change, the metal complexing agent is non-labile (i.e., undergoes ligand exchange very slowly). Further detail with regard to controlling the lability of a complexing agent is set forth in Cotton, particularly in Chapter 28, which is hereby incorporated by reference.

The combinatorial libraries described and used above can be prepared by the following method to which the present invention also relates. In this method, one part (by mole) of a complexing agent, Z (or a salt thereof), is contacted with m¹ parts of A¹ and mⁱ parts of Aⁱ. A¹ and each Aⁱ are non-biopolymer ligands, examples of which are described hereinabove; i is an integer from 2 to k; k is an integer equal to or greater than 3 and represents the number of members in the group of different non-biopolymer ligands from which the non-biopolymer ligands are selected; and the sum of m¹ and Σm¹ from i=2 to k equals n. For example, where the ligands are selected from a group of 4 different ligands (i.e., k=4), these ligands can be identified as A¹, A², A³, and A⁴. Each of A¹, A², A³, and A⁴, are present in amounts (expressed in parts by mole) of m¹, m², m³, and m⁴, respectively, and the sum of m¹, m², m³, and m⁴ is equal to n. Generally, the amounts of the different non-biopolymer ligands in the reaction mixture will be substantially the same. Illustratively, returning to the above example and assuming that the number of ligands which bind to each complexing agent is 2, each of m¹, m², m³, and m⁴ can be about 0.5. The complexing agent and non-biopolymer ligands are contacted under conditions effective to form a substantially statistical mixture of complexes. Suitable conditions are those which permit equilibriums to be established between the various ligands and the complexing agent. Typically, this involves carrying out the reaction at a temperature of from about 0° C. and 100° C., preferably about room temperature, for a time sufficient for equilibration to occur (e.g., about 10 minutes to about 5 days, preferably about 1 to about 2 hours). Either or both of the ligands and complexing agent can be present as a suspension or as a solution, but the latter is preferred. Although it is not generally necessary, the statistical proportions of the various complexes in the resulting combinatorial library can be calculated based on the equilibrium constants of Z and each different ligand and the proportions of the different ligands present in the mixture.

The combinatorial libraries described and used above can also be prepared by the following alternative method to which the present invention also relates. In this method, a complex, Z(A¹)ₙ, is contacted with two or more complexes Z(Aⁱ)ₙ under conditions effective to form a substantially statistical mixture of Z(Aⁱ)ₙ. A¹ and each Aⁱ are non-biopolymer ligands; i is an integer from 2 to k; and k is an integer equal to or greater than 3 and represents the number of members in the group of different non-biopolymer ligands from which the ligands A¹ and Aⁱ are selected. In this embodiment, each of the n A¹'s in Z(A¹)ₙ is the same, and each of the n Aⁱ's in each Z(Aⁱ)ₙ is the same. The Z(A¹)ₙ and Z(Aⁱ)ₙ complexes are contacted under conditions effective to form a substantially statistical mixture of complexes. Suitable conditions are those which permit the ligands on the Z(A¹)ₙ and Z(Aⁱ)ₙ to re-equilibrate. Such conditions include carrying out the reaction at a temperature of from about 0° C. and 100° C., preferably about room temperature; carrying out the reaction in a common solvent, for example, water or a buffered aqueous solution; and carrying out the reaction for a time sufficient for re-equilibration to occur (e.g., about 10 minutes to about 5 days, preferably about 1 to about 2 hours). Either or both of the Z(A¹)ₙ and Z(Aⁱ)ₙ can be present as a suspension or as a solution, but the latter is preferred. Although it is not generally necessary, the statistical proportions of the various complexes in the resulting combinatorial library can be calculated based on the equilibrium constants of Z and each different ligand and the proportions of the various Z(A¹)ₙ and Z(Aⁱ)ₙ initially in the mixture.

The following examples are provided to illustrate embodiments of the present invention but are by no means intended to limit its scope.

### EXAMPLES

### Example 1

### General Materials and Methods

Salicylaldehyde, 3-methoxypropylamine, 3-hydroxypropylamine, 2-aminoethylpyridine, N-methyl 2-aminoethyl pyrrolidine, 2-aminomethylfuran, (4-fluoro)phenethylamine, trifluoroacetic acid, 2-naphthoyl chloride, and methylene chloride were obtained from Aldrich chemical company and used without further purification. Triethylamine was obtained from J. T. Baker Corp. and distilled over calcium hydride under nitrogen atmosphere prior to use. HEPES and phosphate buffered saline (“PBS”) buffers were prepared according to standard protocols from materials purchased from Sigma chemical company. Deionized water was subject to redistillation in an all-glass apparatus prior to use. Zinc chloride and zinc acetate were used as obtained from J. T. Baker Corp. Oligo(dT)-cellulose resin, oligo(dA), and poly(dA-dT) were obtained from Pharmacia Biochem. Unfunctionalized cellulose for control experiments was used as obtained from Sigma chemical company. HPLC data on derivatized amines were obtained using either a Beckman 112 ternary gradient HPLC system equipped with an Alcott fixed-fill autosampler, a Whatman EQ-C18 reverse-phase column (4.6×300 mm), and UV detection, or a Hewlett-Packard 1050 quaternary HPLC equipped with an autosampler, Hamilton RP-1 reverse-phase column (4.6×150 mm), and UV detection. LC-MS was carried out using a Hewlett-Packard Series 1100 MSD, using electrospray ionization in positive ion mode and a Whatman EQ-C18 reverse-phase column. Mass spectral analysis of zinc complexes was performed by direct injection (bypassing the column) of samples into the Hewlett-Packard Series 1100 MSD, with electrospray ionization operating in positive ion mode. UV-Vis spectra and kinetics experiments were carried out on a Shimadzu 1601-PC spectrophotometer at ambient temperature (23° C. +/−1° C.).

### Example 2

### Preparation of DNA Affinity Resins

For each sample, 15 mg oligo(dT) cellulose was weighed into a 2 ml Bio-Rad BIOSPIN™ polypropylene chromatography column equipped with a glass frit. The resin was then taken up in 750 μl HEPES buffer (10 mM HEPES, 100 mM KC1, pH 6.8). 0.5 A₍₂₆₀₎ units of oligo(dA) were then added in 50 μl H₂O and allowed to incubate on the resin for one hour at ambient temperature prior to the addition of library samples. During this pre-incubation period, as well as throughout the remainder of the experiments, gentle agitation was provided by rotating the samples on a Barnstead-Thermolyne LAB QUAKE™ rotary shaker. In a similar manner, 15 mg of unfunctionalized cellulose for each control experiment was allowed to soak in 800 μl HEPES buffer at ambient temperature for one hour prior to the addition of library samples.

### Example 3

### Preparation of Libraries

Amines 1 to 6, having the following formulae:

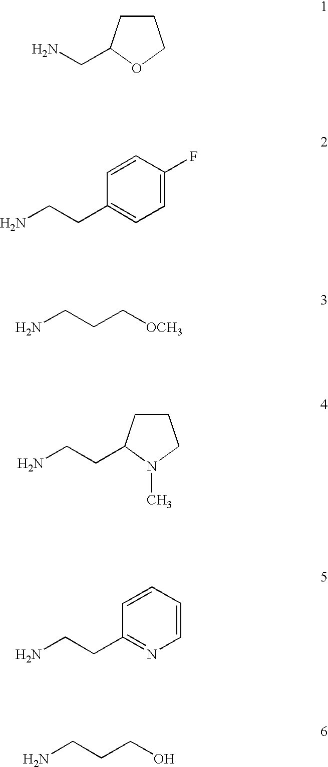

were incubated with salicylaldehyde. Since amines react with the salicylaldehyde to reversibly form imines, the following equilibria are established:

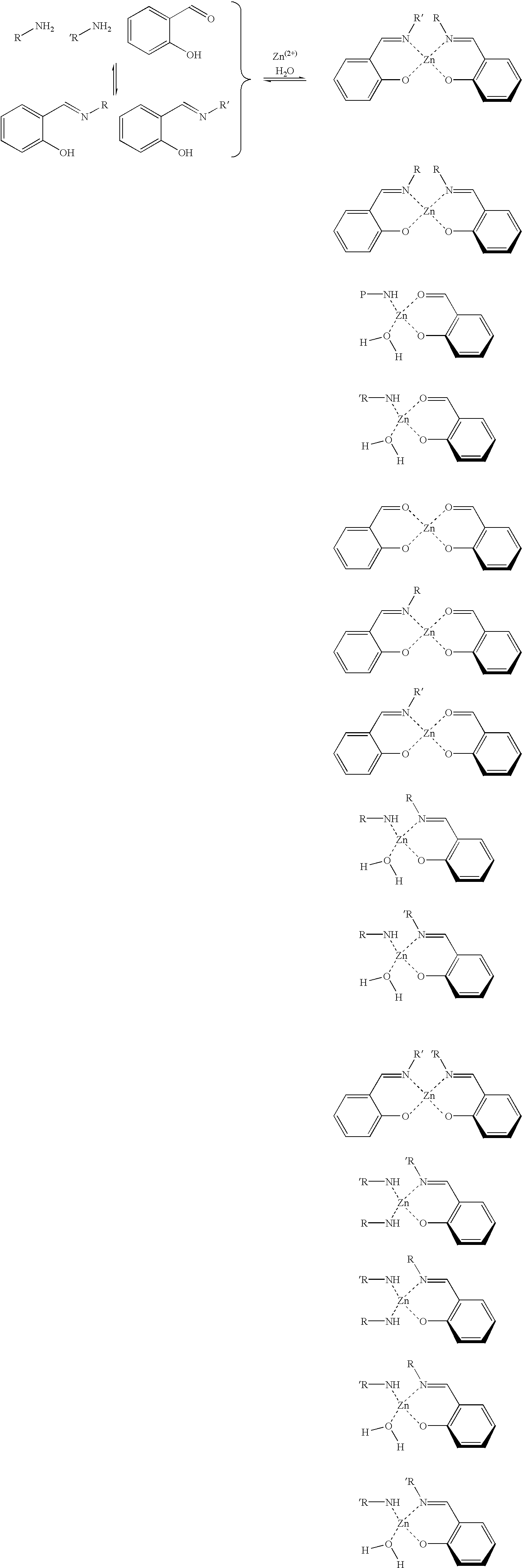

As one skilled in the are will note, the complexes shown above represent only a subset of the complexes that would be present in this equilibrium. Samples were prepared in triplicate from stock solutions as indicated in Table 1 to provide a total volume of 450 μl. An identical set of samples was prepared for control experiments. In each case, libraries were allowed to incubate at ambient temperature for one hour prior to addition to the DNA-cellulose or control cellulose resin. Additional sets of library samples were prepared according to lines C and D of Table 1 for control experiments conducted in the absence of DNA-cellulose or unfunctionalized cellulose resin.

TABLE 1
Library Samples, “High” Amine Concentration

| | Concentration | Zn (2+) | Salicylaldehyde |
|---|---|---|---|
| Sample Set | of each amine | Concentration | Concentration |
| A | 164 | 0 | 82 |
| B | 164 | 41 | 0 |
| C | 164 | 41 | 82 |
| D | 164 | 82 | 82 |

¹All samples were prepared in triplicate, as described in the text. Concentrations are in micromolar, and reflect the final concentration obtained following addition of the library mixture to the DNA-cellulose or control resin.

### Example 4

### Incubation of Libraries With Affinity Resins

Following the pre-incubation period, each sample was added to either a DNA-cellulose or unfunctionalized cellulose column as appropriate. These samples were then allowed to incubate on the resin with gentle agitation at ambient temperature for six hours. Solutions were then eluted from the resin, frozen, and lyophilized prior to derivatization and analysis as described below. Each resin sample was resuspended in 1 ml H₂O and agitated for 24 hours; these wash solutions were then eluted from the resin, frozen, and lyophilized prior to derivatization and analysis as described below.

### Example 5

### Derivatization of Samples

Each sample was resuspended in 1.0 ml of a 50% trifluoroacetic acid solution in dichloromethane, vortexed, and allowed to react for one hour. Solvent was removed under reduced pressure. To each sample of dried material was then added a solution of 0.017 mmol 2-naphthoyl chloride and 200 μl triethyl amine in 0.8 ml methylene chloride. After allowing the reaction to proceed for 10 hours, solvent was removed under reduced pressure. Each crude sample was prepared for HPLC analysis by dissolution in 700 μl of a 20% H₂O −80% CH₃CN solution.

### Example 6

### HPLC Analysis of Samples

In order to separate and identify as many constituents of each sample as possible, a minimum of two HPLC elutions were performed for each sample. In the first, a 30 minute linear gradient of 30% to 100% CH₃CN in H₂O −0.1% trifluoroacetic acid was applied. In the second analysis, a linear gradient of 30% to 100% CH₃CN in 10 mM NH₄OAc (pH 4.5) was applied. Elutions were monitored at 230 nm. HPLC peaks were assigned by comparison with retention times obtained for standard solutions.

### Example 7

### Results

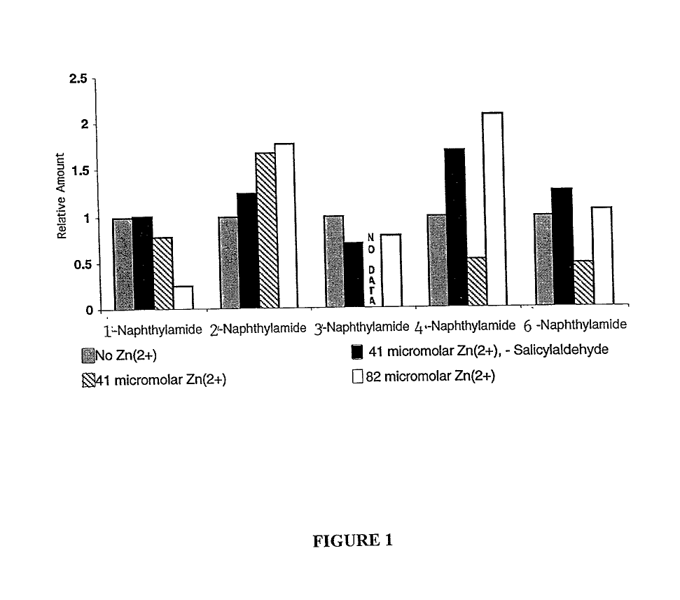

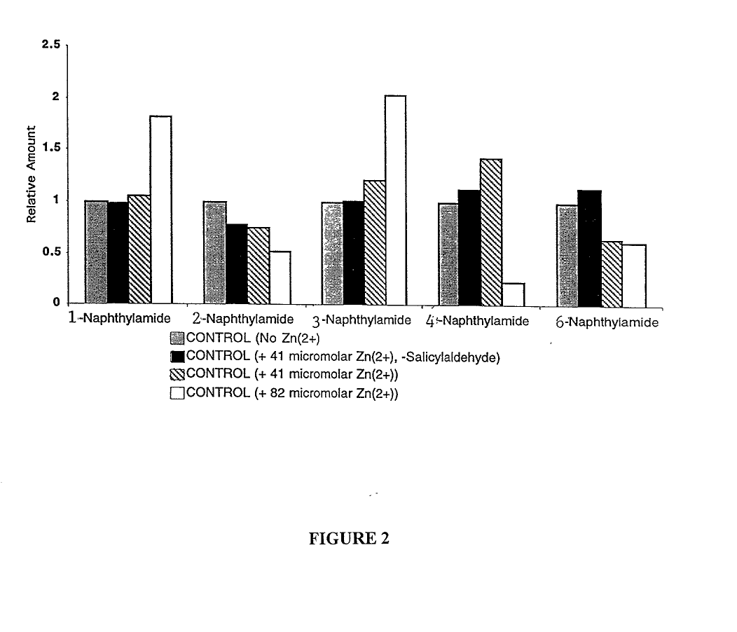

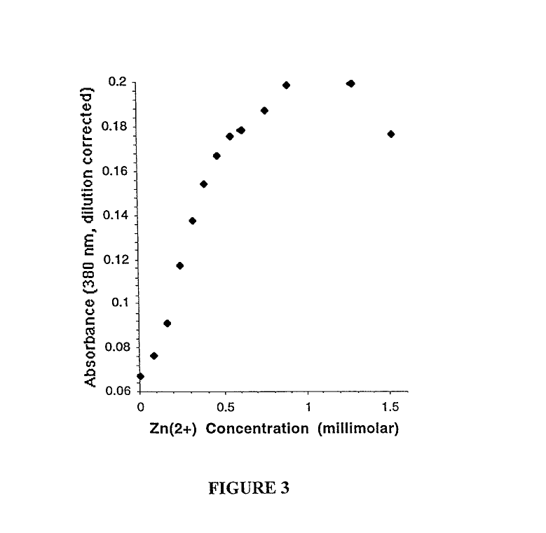

Average results for derivatized amines from initial elutions are shown in FIG. 1. For each case, areas for eluted peaks in the set of zinc-free samples (164 μM each amine, 82 μM salicylaldehyde, no Zn²⁺) were used to normalize all other values. A significant reduction in the amount of derivatized N-methyl 2-aminoethyl pyrrolidine (4) was observed from the three replicate experiments containing both salicylaldehyde and 41 μM Zn²⁺. Similarly, a large (i.e.,>50%) reduction in the amount of 3-hydroxypropylamine (6) was observed under these conditions. An unidentified impurity unfortunately obscured a peak corresponding to 5-naphthylamide, preventing the evaluation of the role of 5 in the library. Comparison with the initial elutions from unfunctionalized cellulose resin (FIG. 2) indicates similar levels of retention of 6 but not 4. A slight (ca. 20%) reduction in the amount of 2-aminomethylfuran (1) in salicylaldehyde and 41 μM Zn²⁺-containing libraries eluted from DNA-cellulose resin was observed. This may indicate a slight affinity of complexes containing 1 for DNA. These data indicate that zinc complexes incorporating salicylaldehyde and N-methyl 2-aminoethylpyrrolidine (4) have the highest affinity for oligo (dA•dT) at the relative concentrations tested. Libraries containing higher (82 μM) concentrations of Zn²+ gave markedly different results from those incorporating 41 μM Zn²⁺. Most notably, a significant reduction in the amount of 4 retained on the column was observed. One likely explanation for this observation is that the added zinc changes the population of complexes present in the library. However, it is not known at present what the precise structural reasons for this might be. As shown in FIG. 3, a strong dependence of the UV spectra of mixtures of salicylaldehyde and N-methyl 2-aminoethyl pyrrolidine (4) in HEPES buffer on the concentration of Zn²⁺ has been observed. However, the concentrations of Zn²⁺ evaluated in these UV titrations which caused the inverse parabolic variations observed were 10-fold higher than those employed in the present affinity experiments.

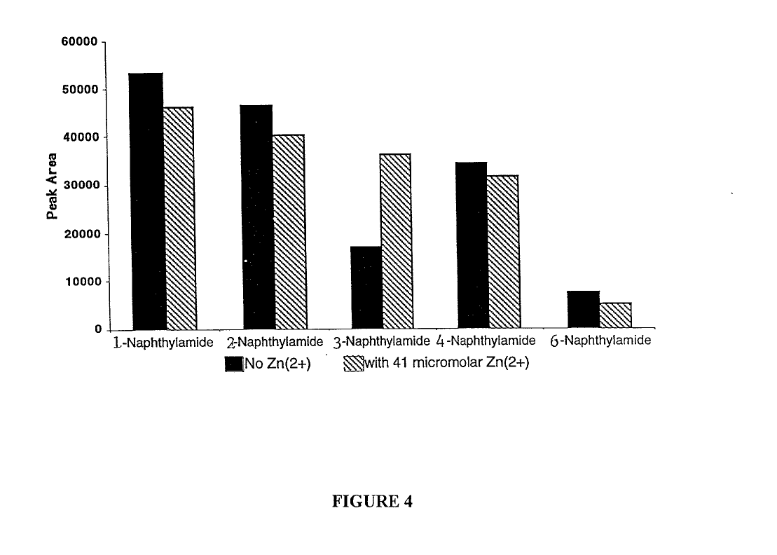

To verify that the presence of zinc was not producing artifacts in the ability to derivatize samples, control libraries, 164 μM in each amine, 82 μM in salicylaldehyde, and 0 or 41 μM in Zn²⁺, were prepared. After allowing these libraries to incubate in solution (i.e., without contact with any sort of resin) for 6 hours, the libraries were lyophilized, derivatized, and analyzed in an identical manner to samples from selection experiments. As shown in FIG. 4, differences in peak volumes for libraries prepared in the presence or absence of Zn²+ are less than 10%, with the exception of 3-methoxypropylamine (3). In each case, the differences could be due to experimental error, participation of Zn²+ in the acylation reaction, or by Zn²+-accelerated partial reversion by hydrolyzed complexes to imines prior to acylation. These results also suggest that differences in peak volumes for derivatized amines that are greater than 10% are probably significant, with the exception of 3-methoxypropylamine.

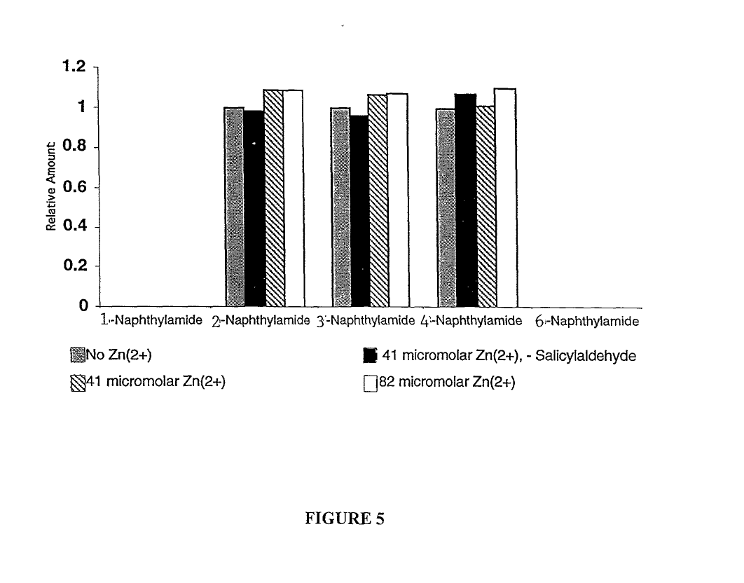

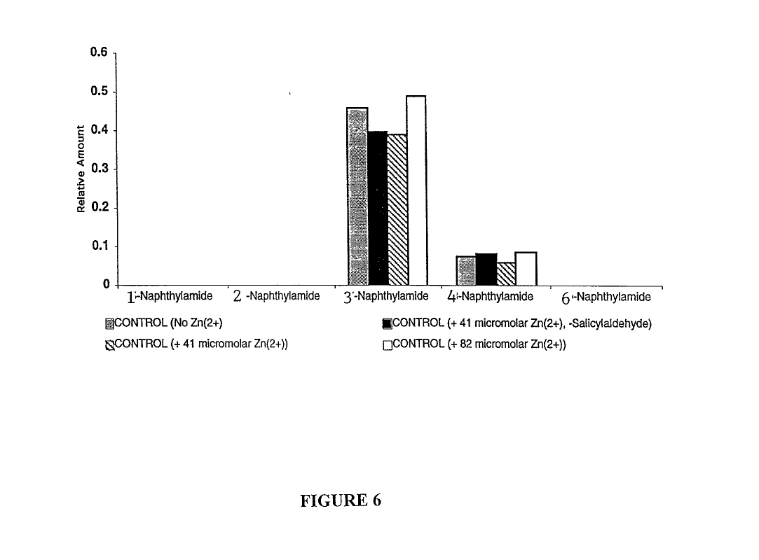

A potentially more desirable method of identifying the library constituents with the highest affinity would be to remove them from the receptor in some manner following the initial elution of “non-binding” components for subsequent derivatization and analysis. It was assumed that simply washing the DNA resin with water would be sufficient to denature the receptor, since low-salt conditions are known to cause DNA melting. However, only small amounts of derivatized amines from wash solutions were observed, with no experimentally significant differences among experiments or between DNA-cellulose (FIG. 5) and control cellulose (FIG. 6) resins.

### Example 8

### Alternative Method

Rather than forming the imine constituents of the ligands from a mixture of salicylaldehyde and amines, the imines can be pre-formed, as set forth below.

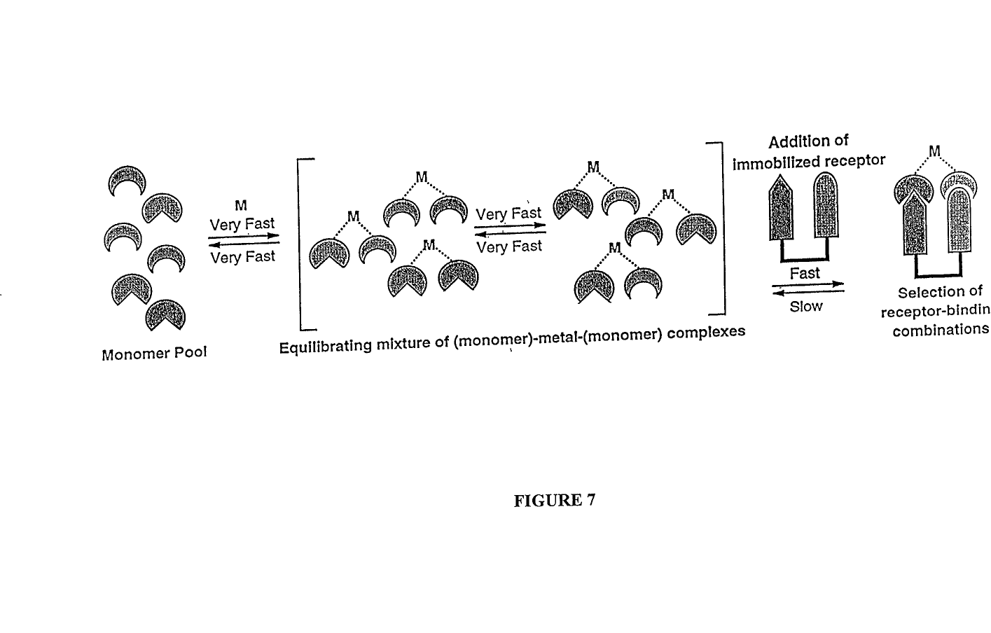

The experimental design is shown in schematic form in FIG. 7. Introduction of a transition metal salt (“M”) to a pool of compounds (“monomers”) capable of forming coordination compounds with M should initiate a series of equilibria among various combinations of complexes. Assuming ligand exchange is reasonably fast, this equilibration permits all combinatiorally possible coordination complexes to be populated. By adding a receptor covalently linked to a solid support (an affinity resin), however, an additional equilibration ensues involving the binding of coordination compounds to the receptor with varying degrees of affinity. Since some compounds might be expected to bind with higher affinity, those compounds would be depleted from the pool of equilibrating complexes. By simple mass balance rules, the equilibrium would then have to shift in favor of compounds that bind to the receptor, effectively utilizing the receptor as a catalyst for the synthesis of its own ligand.

While this concept could easily be applied to any type of biopolymer or other “receptor”, double-stranded DNA was chosen as the initial target, for a number of reasons. First, the design and synthesis of sequence-selective DNA-binding agents is a problem of continuing fundamental and medical importance. (For example, see Trauger et al., J. Am Chem. Soc., 118:6160-6166 (1996) and references therein, which are hereby incorporated by reference.) Second, the ladder-like structure of DNA is ideally suited to the application of a modular, self-assembly strategy. Finally, several oligonucleotide affinity resins are available commercially, allowing the experimental details of initial selection strategies to be determined without the need for costly oligonucleotide and affinity resin synthesis. As “monomers”, the readily available salicylaldimines 7, which have the following formula:

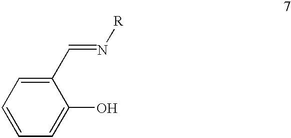

were chosen. Salicylaldimines are well known to form coordination complexes with a wide variety of transition metals (Gamovskii et al., Coord. Chem. Rev., 126:1-69 (1993), which is hereby incorporated by reference) and a substantial amount of structural information about these complexes is available. Because of its tetrahedral coordination geometry with most salicylaldimines (Dreher et al., Naturforsch. B., 42:707-712 (1987), which is hereby incorporated by reference) and compatibility with nucleic acids, divalent zinc was used as the metal for initial studies.

Six salicylaldimines (8-13) displaying a variety of sidechain functionality and having the formulae:

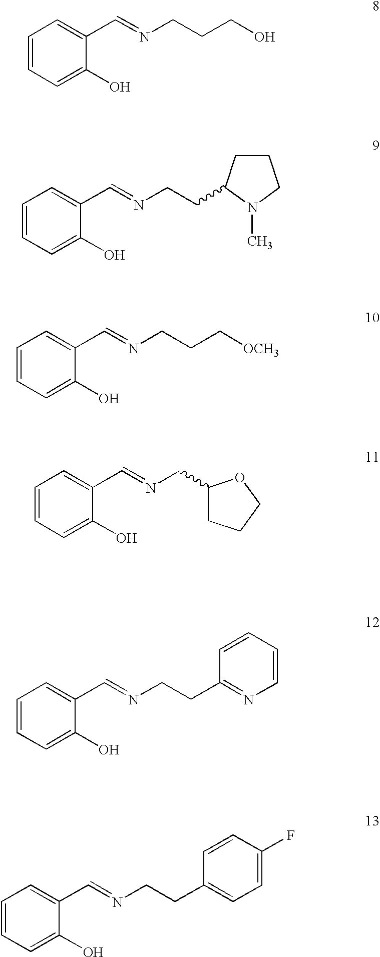

were synthesized condensation of salicylaldehyde and commercially available amines. The synthesis and characterization of 8 have been previously described in Inouye, S. Chem. Pharm. Bull., 15:1540-1546 (1967), which is hereby incorporated by reference. The synthesis and characterization of compound 10 have been previously described in Duran et al., Synth. React. Inorg. Met.-Org. Chem., 17:681-683 (1987), which is hereby incorporated by reference, and in Ramesh et al., J. Chem. Soc. Dalton, pp. 83-86 (1992), which is hereby incorporated by reference. The synthetic procedures for 8, 10, 11, and 13 are set forth below in Example 10. Given that compounds 9 and 11 were synthesized in racemic form, a combinatorial library formed from these six salicylaldimines could be expected to provide a maximum of 36 unique bis(salicylaldiminato)zinccomplexes. These compounds were chosen in part because millimolar solutions of each could be prepared in buffered aqueous solution with 1% DMSO as cosolvent, a finding important to their successful utilization in a combinatorial library.

Although the ability of bis(salicylaldiminato)-zinc(Blackborow et al., J. Chem. Res., 119 (1978), which is hereby incorporated by reference) and -nickel (Schepartz et al., J. Am.Chem. Soc., 111:5976-6977 (1989), which is hereby incorporated by reference) complexes to self-assemble in halogenated solvents is well known, we felt it was important to verify that this could also occur in aqueous solution before attempting to construct a -combinatorial library. Therefore, the dependence of the proton NMR spectrum for 12 on ZnCl₂ concentration in D₂O was examined. Characteristic of slow-exchange phenomena, substantial line broadening of aromatic, and, to a lesser extent, side-chain resonances, is observable on addition of substoichiometric quantities of ZnCl₂. In addition, chemical shifts change markedly. All peaks coalesce into a single set of sharply defined resonances as excess zinc is added. These observations do not provide high-resolution structural information about these complexes. Therefore, it is conceivable that the structure in solution is an addition complex (containing two salicylaldimines coordinated to ZnCl₂) rather than a chelate (see Bottino et al., Zeitschrift fur Kristallographie, 187:71-77 (1989), which is hereby incorporated by reference). However, these observations do indicate that complexation between the salicylaldimine and Zn²+ occurs, providing a single coordinated species. It was also observed that the addition of ZnCl₂ to a solution of an amine and salicylaldehyde in water can induce imine formation followed by complexation. This suggests that a three-stage equilibrium process could be constructed.

A serious potential problem with the selection and amplification of a single compound from an equilibrating mixture of complexes is that of identifying the desired high-affinity species. First, if the selection is not complete for a single compound, the separation and identification of a potentially large number of complexes presents a significant analytical challenge. Furthermore, since reequilibration of complexes can occur once the affinity resin is removed, it might be expected that binding information would be lost. However, we found that hydrolysis of mixtures of complexes with trifluoroacetic acid followed by neutralization and derivatization with excess 2-naphthoyl chloride provided a mixture of amide derivatives that was readily separable by standard reverse-phase HPLC. Application of this technique to the eluent from an affinity column would permit deconvolution of the library to provide information about the identity of binding complexes.

### Example 9

### Results for Alternative Method

With a series of monomers, verification of self-assembly in aqueous solutions, and a separation method in hand, the experimental tools were in place to conduct an affinity selection and amplification experiment. A standard solution of 0.5 mM 8-13 (total concentration 3 mM in “monomer”) was prepared in 10 mM Tris•HCl, 100 mM KCl, 1% DMSO, pH 7.5. To determine the effect of zinc on the observed selection, samples were prepared either with or without 26 MM ZnCl₂. Samples were allowed to incubate for one hour to allow an equilibrium mixture of complexes to form, and the latter were then individually added to affinity columns prepared from 30 mg of commercial poly(dT)-cellulose resin (Pharmacia Biotech) preincubated with one A₂₆₀ unit (approximately 7 nmol) of oligo (dA)₁₂₋₁₈(Oligo (dA)₁₂₋₁₈ as obtained from Pharmacia Biotech is a mixture of homopolymeric, single-stranded DNA of length 12 to 18) in 10 mM Tris•HCl, 100 mM KCl, pH 7.5 for formation of double-stranded DNA (giving an approximate, average complex:base pair ratio of 85:1). Following a two-hour incubation of the libraries on the resin, solutions were eluted, lyophilized, and derivatized as described above.

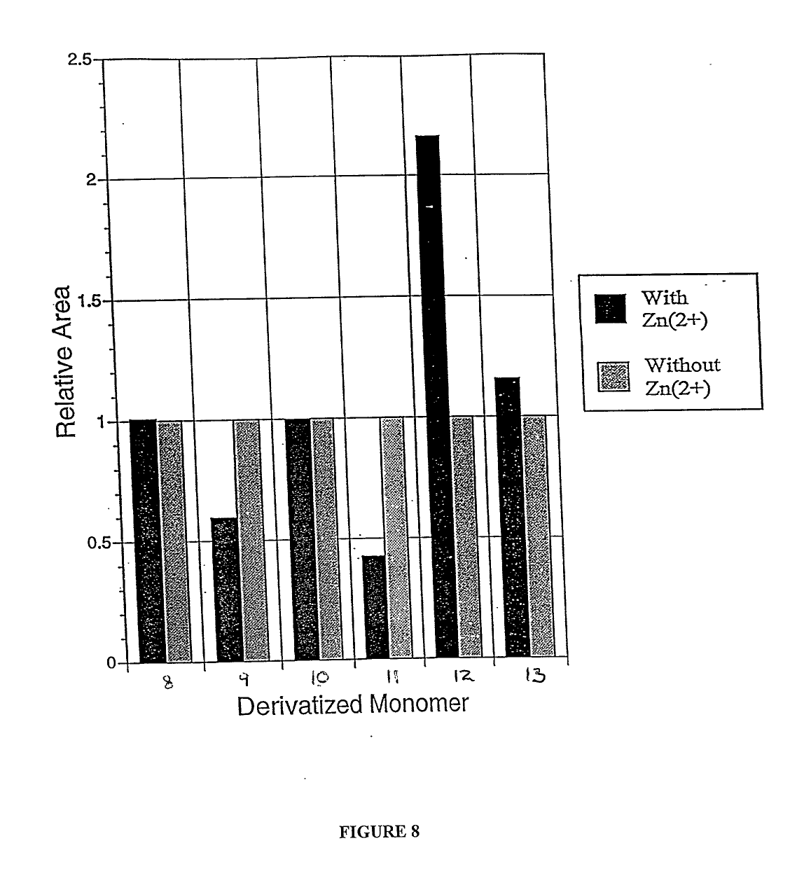

FIG. 8 shows the results of affinity selection and amplification of our initial self-assembled combinatorial library. With data normalized to values observed in the absence of zinc, it is clear that monomers 9 and 11 are most strongly retained on the affinity column in the presence of zinc (i.e., less of these monomers is observed following elution and derivatization). The observation that twice as much of derivatized monomer 12 is eluted from the affinity column in the presence of zinc as in the absence of zinc suggests that this monomer participates in some kind of binding interaction with double-stranded oligo(dA)-oligo(dT)in the absence of zinc that is not available to either the 12-Zn-12 complex, or other complexes incorporating monomer 7. These results would then require deconvolution of three possibilities for the strongest binding complex: 9-Zn-9, 11-Zn-11, and 9-Zn-11 (including all stereoisomers) would all be candidates. However, results of control experiments rendered this question moot.

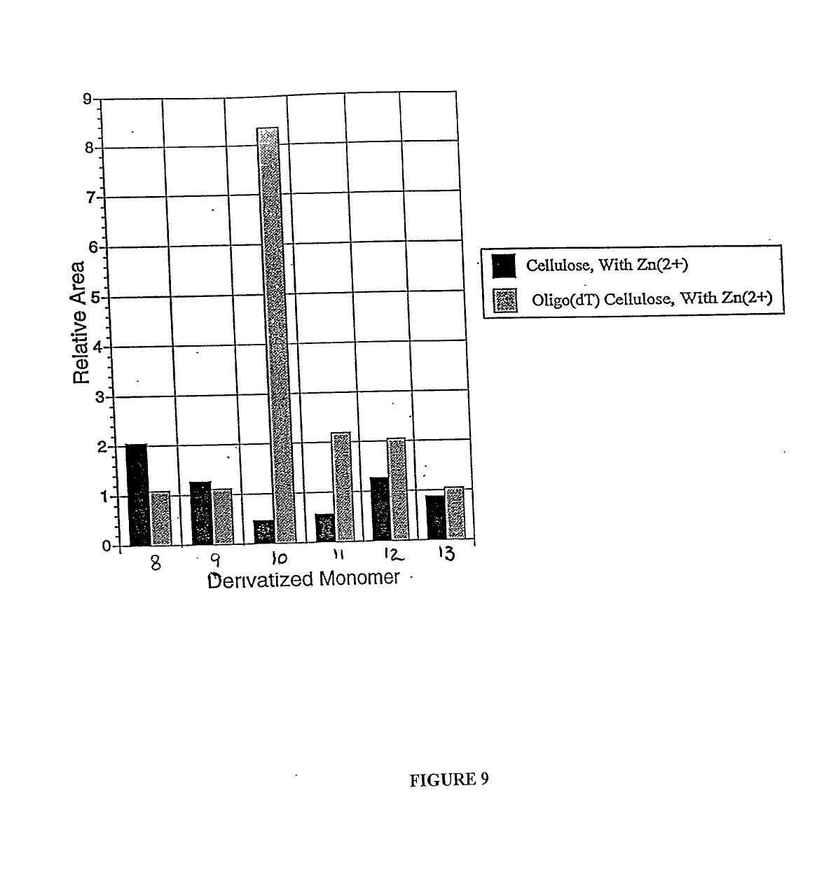

To ensure that selections observed were based on differential affinity for double-stranded DNA, rather than interactions with single-stranded DNA or with the underlying cellulose support, control experiments were conducted using single-stranded oligo(dT)-cellulose, and cellulose alone, under conditions identical to those for the double-stranded experiment. As shown in FIG. 9, monomer 9 is retained on cellulose or oligo(dT)-cellulose to the same extent in the presence or absence of zinc, while 11 is more strongly retained on cellulose in the presence of zinc. This suggests that two out of the three possibilities for the strongest binding complex to double-stranded DNA (11-Zn-11 and 1 1-Zn-9) do not need to be considered, leaving 9-Zn-9 as likely the tightest DNA-binding complex. Other monomers are generally retained more strongly to single-stranded oligo(dT) cellulose in the absence of zinc.

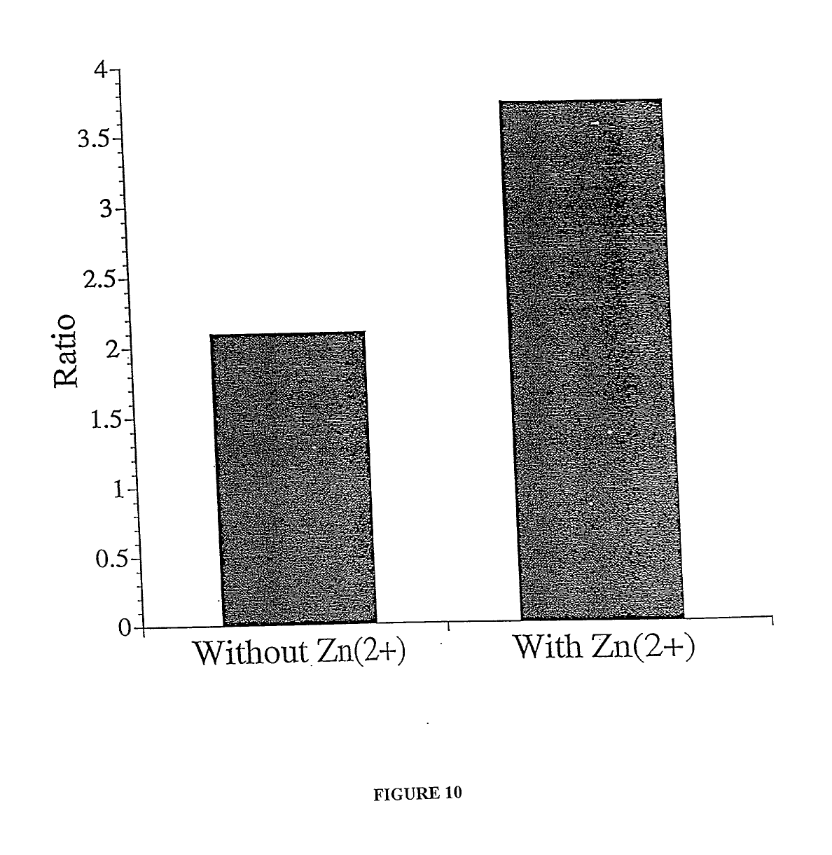

In order to verify that the results of the affinity selection experiment accurately reflected differences in binding affinities, UV binding titrations (as described in Krugh, Proc. Natl. Acad. Sci. USA, 69:1911-1914 (1972)) were conducted for homodimeric complexes 10-Zn-10 (a presumed weak- or non-binding complex) and 9-Zn-9 (a presumed strong-binding complex). Since both of these complexes have strong UV absorbances in the range of 290-350 nm while poly(dA)-poly(dT) does not, the concentration of complex was held constant in the presence of varying concentrations of DNA in each case. Using commercially available poly(dA)-poly(dT), saturable binding was observable in each case. While any analysis of binding constants for these complexes is complicated by the homopolymeric, variable-length nature of the DNA used (providing multiple, identical binding sites), 9-Zn-9 bound to DNA with an apparent K_{D} of 1.11 micromolar, significantly stronger than 10-Zn-10 (27.8 micromolar). A simple affinity selection experiment conducted as described above, but utilizing only monomers 9 and 10, further corroborated these observations. As shown in FIG. 10, the ratio of derivatized 10:9 eluted following incubation on double-stranded oligo(dT)-cellulose doubles on addition of zinc.

### Example 10

### Preparation of 8, 10, 11, and 13

All solvents, salicylaldehyde, and amines were obtained from Aldrich Chemical Company and were used without further purification. NMR spectra were recorded on a Nicolet/GE QE-300 FT spectrometer operating at 300 (¹H) or 75 (¹³C) MHz. IR spectra were recorded using a Perkin-Elmer model 1600 FTIR. High-resolution mass spectra (HRMS) analyses were carried out by the UC-Riverside Mass Spectrometry Facility, Riverside, Calif.

Compound 9 was prepared as follows. To a solution of salicylaldehyde (3.0 mmol) and 2-(2-aminoethyl)-1-methyl pyrrolidine (3.0 mmol) in dichloromethane was added an excess (1 ml, or approximately 2.6 equivalents) triethylamine. Reaction was allowed to proceed with stirring at room temperature for two hours. Solvent and triethylamine were then removed in vacuo to provide essentially pure 9 in quantitative yield. ¹H NMR (CDCl₃, 300 MHz): δ13.15 (s, 1H), 8.39 (s, 1H), 7.3 (m, 2H), 6.9 (m, 2H), 3.62 (m, 2H), 3.1 (m, 1H), 2.34 (s, 3H), 2.1 (m, 4H), 1.7 (m, 4H). ¹³CNMR (CDCl₃, 75 MHz): δ164.6, 161.2, 132.1, 131, 118.8, 118.4, 117, 64.2, 57.2, 57.1, 40.5, 35.1, 30.8, 22. IR (thin film): 3788.7, 3695.5, 3661.2, 3636.2, 2941.2, 2840.0, 2776.5, 2666.1, 2360.0, 2341.5, 1725.3, 1709.5, 1688.4, 1664.0, 1632.4, 1581.0, 1549.5, 1497.1, 1460.4, 1415.5, 1370.4, 1344.2, 1280.3, 1212.4, 1150.9, 1117.2, 1031.6, 970.1, 891.3, 847.6, 755.8, 736.2, 668.0. HRMS: calc. 233.1528; obs. 233.1647.

As with 9, 10 was prepared using 3-methoxypropylamine. ¹H NMR (CDCl₃, 300 MHz): δ13.57 (s, 1H), 8.19 (s, 1H), 6.55 (m, 2H), 6.4 (d, 1H, J=9 Hz), 6.38 (t, 1H, J=9 Hz), 2.9 (t, 2H, J=7.5 Hz), 2.8 (t, 2H, J=7.5 Hz), 2.78 (s, 3H), 20 (q,2H, J=7.5 Hz). ¹³C NMR (CDCl₃, 75 MHz): δ165, 161, 135, 133, 119, 118, 116,70, 59, 56, 32. IR (thin film): 3845.6, 3788.5, 3695.5, 3681.7, 1660.9, 3638.0, 2924.1, 2870.0, 1725.3, 1709.8, 1689.2, 1657.4, 1632.5, 1581.3, 1549.6, 1529.2, 1497.5, 1460.8, 1414.6, 1384.6, 1337.6, 1279.6, 1202.0, 1151.0, 1118.1, 1031.3, 974.1, 849.0, 756.4, 736.4, 640.1. HRMS: calc. 194.1055; obs. 194.1187

As with 9, 11 was prepared from 2-aminomethylfuran. ¹H NMR (CDCl₃, 300 MHz): δ13.19 (s, 1H), 8.40 (s, 1H), 7.3 (m, 2H), 6.9 (m, 2H), 4.2 (m, 1H), 3.8 (m, 4H), 1.95 (m, 4H). ¹³CNMR (CDCl₃, 75 MHz): δ166.31, 161.70, 132.64,131.69, 119.28, 118.89, 117.44, 78.61, 68.86, 63.91, 29.59, 26.29. IR(thin film): 3947.8,3256.7,3055.7, 2871.7, 2733.4, 2661.7, 2061.3, 1945.4, 1912.4, 1790.0, 1633.1, 1580.7, 1496.2, 1461.8, 1416.4, 1365.4, 1315.8, 1281.5, 1246.9, 1209.6, 1151.2, 1116.1, 1074.6, 1046.3, 1026.7, 996.6, 966.4, 922.1. HRMS: calc. 206.1182; obs. 206.1185

As with 9, 13 was prepared from 4-fluorophenethylamine. ¹H NMR (CDCl₃, 300 MHz): δ13.39 (s, 1H) 8.1 (s, 1H), 7.35 (m, 1H), 7.2 (m, 2H), 7.0 (m, 2H), 6.9 (m, 2H), 3.8 (t, 2H, J=7.5 Hz), 3.0 (t, 2H, J=7.5 Hz). ¹³C NMR (CDCl₃, 75 MHz):δ168, 164, 161, 159, 135, 133, 132, 130, 118, 117.5, 115, 114.5, 68, 61, 36.5. IR (thin film): 3044.6, 2926.6, 2852.4, 2732.1, 2662.9, 1887.1, 1633.2, 1607.6, 1580.0, 1509.6, 1460.7, 1415.6, 1376.4, 1342.1, 1279.9, 1221.2, 1156.8, 1116.8, 1098.2, 1073.7, 1053.0, 1016.1, 9687.6, 894.1, 870.1, 824.5, 778.1, 757.0, 736.3, 724.3, 705.5, 653.1, 640.8, 573.8,542.5, 509.5,481.9,467.6. HRMS:calc. 244.1011; obs 244.1129.

### Example 11

### NMR Titration of ZnCl₂ into 10

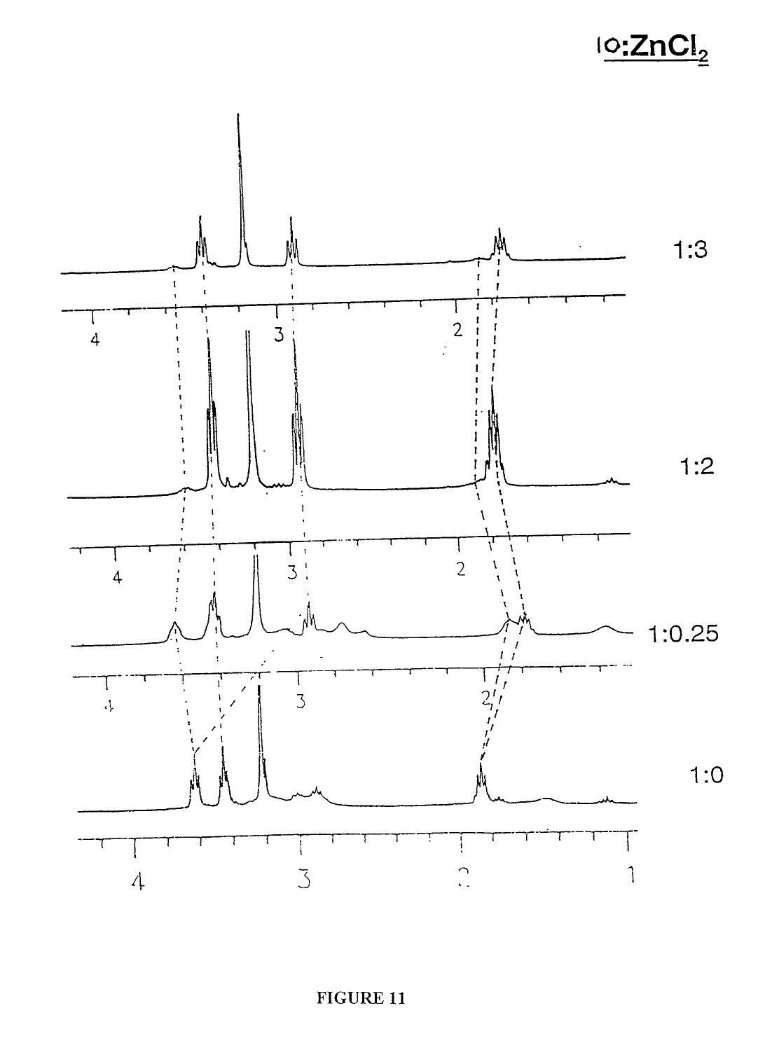

A 97 mM solution of 10 in deuterated buffer (PBS, pH 7.4) was prepared, and the ¹H NMR spectrum measured at 300 MHz, 23 ° C. Aliquots of a 1M solution of ZnCl₂ were then added to permit measurement of the NMR spectrum at 1:0.25, 1:2, and 10 1:3 molar ratios of 10:Zn² . Plots of the observed NMR spectra are shown in FIG. 11.

Although the invention has been described in detail for the purpose of illustration, it is understood that such detail is solely for that purpose, and variations can be made therein by those skilled in the art without departing from the spirit and scope of the invention which is defined by the following claims.

What is claimed:

1. A combinatorial library comprising
a plurality of at least six different complexes, each formed of at least one complexing agent and at least two non-biopolymer ligands that are reversibly bonded to the complexing agent, wherein each different complex in said library has different ligands bonded to the complexing agent.

2. A combinatorial library according to claim 1, wherein each of said plurality of complexes has the formula Z(Aⁱ)ₙ, wherein Z is a complexing agent capable of reversibly binding to two or more ligands, each Aⁱ is a non-biopolymer ligand capable of reversibly binding to Z and is independently selected from a group of non-biopolymer ligands having at least three different members, n is the number of A's that are reversibly bonded to Z and is an integer equal to two or greater, and i is an index number for each A and is an integer from 1 to n.

3. A combinatorial library according to claim 2, wherein each of said plurality of complexes has the formula Z(A¹)(A²)(Aⁱ)ₙ₋₂, wherein A¹ and A² are non-biopolymer ligands capable of reversibly binding to Z and are independently selected from a group of non-biopolymer ligands having at least three different members and i is an index number for each A and is an integer from 3 to n.

4. A combinatorial library according to claim 3, wherein said combinatorial library comprises complexes having the formulae Z(B1)(B1)(A¹)ₙ₋₂, Z(B1)(B2)(Aⁱ)ₙ₋₂, Z(B1)(B3)(Aⁱ)ₙ₋₂, Z(B2)(B2)(Aⁱ)ₙ₋₂, Z(B2)(B3) (Aⁱ)ₙ₋₂, and Z(B3)(B3)(Aⁱ)ₙ₋₂; B1, B2, and B3 are different non-biopolymer ligands and are members of the group from which each Aⁱ is selected; and i is an index number for each A and is an integer from 3 to n.

5. A combinatorial library according to claim 2, wherein each of said plurality of complexes has the formula Z(A¹)(Aⁱ)ₙ₋₁, wherein A¹ is a non-biopolymer ligand capable of reversibly binding to Z and is independently selected from a group of non-biopolymer ligands having at least three different members; i is an index number for each A and is an integer from 3 to n; and Z, A¹, and each Aⁱ are selected so that the reactions Z(Aⁱ)ₙ₋₁+A¹→Z(A¹)(Aⁱ)ₙ₋₁ and Z(A¹)(Aⁱ)ₙ₋₁→Z(Aⁱ)ₙ₋₁+A¹ each have a rate constant of greater than about 2 per second.

6. A combinatorial library according to claim 2, wherein at least one of Aⁱ is a DNA intercalator or a major or minor groove DNA binder.

7. A combinatorial library according to claim 2, wherein Z is a transition metal.

8. A combinatorial library according to claim 2, wherein at least one Aⁱ has the formula —A′Z′(Q), Z′ is a second complexing agent identical to or different than Z, A′ is a pluridentate non-biopolymer ligand, and Q is one or more non-biopolymer ligands.

9. A combinatorial library according to claim 3, wherein A¹ and A², taken together, have the formula: 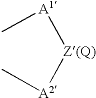
where A^{1′} and A^{2′} are pluridentate non-biopolymer ligands Z′ is a second complexing agent identical to or different than Z, and Q is one or more non-biopolymer ligands so that Z(A¹)(A²)(Aⁱ)ₙ₋₂ has the formula:

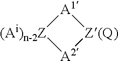

10. A combinatorial library according to claim 1, wherein said combinatorial library comprises a plurality of at least 100 different complexes.

11. A composition comprising:
a combinatorial library according to claim 1, and
a receptor in contact with the combinatorial library, wherein some of the complexes bind preferentially to said receptor.

12. A composition according to claim 11, wherein said receptor is a biological receptor.

13. A composition according to claim 11, wherein said receptor is an immobilized receptor.

14. A composition according to claim 11, wherein each of the plurality of complexes has the formula Z(Aⁱ)ₙ, wherein Z is a complexing agent capable of reversibly binding to two or more ligands, each Aⁱ is a non-biopolymer ligand capable of reversibly binding to Z and is independently selected from a group of non-biopolymer ligands having at least three different members, n is the number of A's that are reversibly bonded to Z and is an integer equal to two or greater, and i is an index number for each A and is an integer from 1 to n.

15. A composition according to claim 14, wherein each of the plurality of complexes has the formula Z(A¹)(A²)(Aⁱ)ₙ₋₂, wherein A¹ and A² are non-biopolymer ligands capable of reversibly binding to Z and are independently selected from a group of non-biopolymer ligands having at least three different members and i is an index number for each A and is an integer from 3 to n.

16. A composition according to claim 15, wherein said combinatorial library comprises complexes having the formulae Z(B1)(B1)(Aⁱ)ₙ₋₂, Z(B1)(B2)(Aⁱ)ₙ₋₂, Z(B1)(B3)(Aⁱ)ₙ₋₂, Z(B2)(B2)(Aⁱ)ₙ₋₂, Z(B2)(B3)(Aⁱ)ₙ₋₂, and Z(B3)(B3)(Aⁱ)ₙ₋₂; B1, B2, and B3 are different non-biopolymer ligands and are members of the group from which each Aⁱ is selected; and i is an index number for each A and is an integer from 3 to n.

17. A composition according to claim 14, wherein each of the plurality of complexes has the formula Z(A¹)(Aⁱ)ₙ₋₁, wherein A¹ is a non-biopolymer ligand capable of reversibly binding to Z and is independently selected from a group of non-biopolymer ligands having at least three different members; i is an index number for each A and is an integer from 3 to n; and Z, A¹, and each Aⁱ are selected so that the reactions Z(Aⁱ)ₙ₋₁+A¹→Z(A¹)(Aⁱ)ₙ₋₁ and Z(A¹)(Aⁱ)ₙ₋₁→Z(Aⁱ)ₙ₋₁+A¹ each have a rate constant of greater than about 2 per second.

18. A composition according to claim 14, wherein at least one of the Aⁱ is a DNA intercalator or a major or minor groove DNA binder.

19. A composition according to claim 14, wherein Z is a transition metal.

20. A composition according to claim 14, wherein at least one Aⁱ has the formula —A′Z′(Q), Z′ is a second complexing agent identical to or different than Z, A′ is a pluridentate non-biopolymer ligand, and Q is one or more non-biopolymer ligands.

21. A composition according to claim 15, wherein A¹ and A², taken together, have the formula: 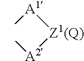
where A^{1′} and A^{2′} are pluridentate non-biopolymer ligands , Z′ is a second complexing agent identical to or different than Z, and Q is one or more non-biopolymer ligands so that Z(A¹)(A²)(Aⁱ)ₙ₋₂ has the formula:

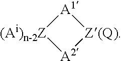

22. A composition according to claim 12, wherein said combinatorial library comprises a plurality of at least 100 different complexes.

23. A method of identifying a combination of non-biopolymer ligands which bind preferentially to a receptor comprising:
providing a combinatorial library according to claim 1;
contacting the combinatorial library with a receptor under conditions effective to preferentially bind a fraction of the plurality of complexes; and
identifying the fraction of the plurality of complexes which are bound preferentially to the receptor.

24. A method according to claim 23, wherein the receptor is a biological receptor.

25. A method according to claim 23, wherein the receptor is an immobilized receptor.

26. A method according to claim 23, wherein each of the plurality of complexes has the formula Z(Aⁱ)ₙ, wherein Z is a complexing agent capable of reversibly binding to two or more ligands, each Aⁱ is a non-biopolymer ligand capable of reversibly binding to Z and is independently selected from a group of non-biopolymer ligands having at least three different members, n is the number of A's that are reversibly bonded to Z and is an integer equal to two or greater, and i is an index number for each A and is an integer from 1 to n.

27. A method according to claim 26, wherein each of said plurality of complexes has the formula Z(A¹)(A²)(Aⁱ)ₙ₋₂, wherein A¹ and A² are non-biopolymer ligands capable of reversibly binding to Z and are independently selected from a group of non-biopolymer ligands having at least three different members and i is an index number for each A and is an integer from 3 to n.

28. A method according to claim 27, wherein said combinatorial library comprises complexes having the formulae Z(B1)(B1)(Aⁱ)ₙ₋₂, Z(B1)(B2)(Aⁱ)ₙ₋₂, Z(B1)(B3)(Aⁱ)ₙ₋₂, Z(B2)(B2)(Aⁱ)ₙ₋₂, Z(B2)(B3)(Aⁱ)ₙ₋₂, and Z(B3)(B3)(Aⁱ)ₙ₋₂, B1, B2, and B3 are different non-biopolymer ligands and are members of the group from which each Aⁱ is selected; and i is an index number for each A and is an integer from 3 to n.

29. A method according to claim 26, wherein each of said plurality of complexes has the formula Z(A¹)(Aⁱ)ₙ₋₁, wherein A¹ is a non-biopolymer ligand capable of reversibly binding to Z and is independently selected from a group of non-biopolymer ligands having at least three different members; i is an index number for each A and is an integer from 3 to n; and Z, A¹, and each Aⁱ are selected so that the reactions Z(Aⁱ)ₙ₋₁+A¹→Z(A¹)(Aⁱ)ₙ₋₁ and Z(A¹)(Aⁱ)ₙ₋₁→Z(Aⁱ)ₙ₋₁+A¹ each have a rate constant of greater than about 2 per second.

30. A method according to claim 26, wherein at least one Aⁱ is a DNA intercalator or a major or minor groove DNA binder.

31. A method according to claim 26, wherein Z is a transition metal.

32. A method according to claim 26, wherein at least one Aⁱ has the formula —A′Z′(Q), Z′ is a second complexing agent identical to or different than Z, A′ is a pluridentate non-biopolymer ligand, and Q is one or more non-biopolymer ligands.

33. A method according to claim 27, wherein A¹ and A², taken together, have the formula: 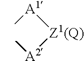
where A^{1′} and A^{2′} are pluridentate non-biopolymer ligands, Z′ is a second complexing agent identical to or different than Z, and Q is one or more non-biopolymer ligands so that Z(A¹)(A²)(Aⁱ)ₙ₋₂ has the formula:

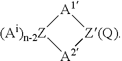

34. A method according to claim 23, wherein the combinatorial library comprises a plurality of at least 100 different complexes.

35. A method according to claim 26, wherein some of Z(Aⁱ)ₙ bind more strongly than others of Z(Aⁱ)ₙ to the receptor.

36. A method according to claim 23, wherein said providing a combinatorial library comprises:
contacting a compound containing a complexing agent with a plurality of at least three different non-biopolymer ligands.

37. A method according to claim 26, wherein said providing a combinatorial library comprises:
contacting one part of a complexing agent, Z, or a salt thereof with m¹ parts of A¹ and mⁱ parts of Aⁱ, wherein A¹ and each Aⁱ are non-biopolymer ligands, i is an integer from 2 to k, k is an integer equal to or greater than 3 and represents the number of members in the group of at least three different non-biopolymer ligands from which the at least two non-biopolymer ligands are selected, and the sum of mⁱ and Σm¹ from i=2 to k equals n under conditions effective to form a substantially statistical mixture of Z(Aⁱ)ₙ.

38. A method according to claim 26, wherein said providing a combinatorial library comprises:
contacting a complex, Z(A¹)ₙ, with two or more complexes Z(Aⁱ)ₙ, wherein A¹ and each Aⁱ are non-biopolymer ligands, i is an integer from 2 to k, and k is an integer equal to or greater than 3 and represents the number of members in the group of at least three different non-biopolymer ligands from which the at least two non-biopolymer ligands are selected.

39. A method of producing a combinatorial library according to claim 2 comprising:
contacting one part of a complexing agent, Z, or a salt thereof with m¹ parts of A¹ and mⁱ parts of Aⁱ under conditions effective to form a substantially statistical mixture of Z(Aⁱ)ₙ, wherein A¹ and each Aⁱ are non-biopolymer ligands, i is an integer from 2 to k, k is an integer equal to or greater than 3 and represents the number of members in the group of at least three different non-biopolymer ligands from which the at least two non-biopolymer ligands are selected, and the sum of m¹ and Σmⁱ from i=2 to k equals n.

40. A method of producing a combinatorial library according to claim 2 comprising:
contacting a complex, Z(A¹)ₙ, with two or more complexes Z(Aⁱ)ₙ under conditions effective to form a substantially statistical mixture of Z(Aⁱ)ₙ, wherein A¹ and each Aⁱ are non-biopolymer ligands, i is an integer from 2 to k, and k is an integer equal to or greater than 3 and represents the number of members in the group of at least three different non-biopolymer ligands from which the at least two non-biopolymer ligands are selected.
